(19) United States
(12) Patent Application Publication
(10) Pub. No.: US 2023/0374063 A1
(43) Pub. Date: Nov. 23, 2023

(54) METHODS FOR REDUCING HOST CELL PROTEIN CONTENT IN PROTEIN PURIFICATION PROCESSES

(71) Applicant: Eli Lilly and Company, Indianapolis, IN (US)

(72) Inventors: Brian David Bowes, Indianapolis, IN (US); Lara Ellen Krebs, Indianapolis, IN (US)

(21) Appl. No.: 18/247,084

(22) PCT Filed: Oct. 4, 2021
(86) PCT No.: PCT/US2021/053318

§ 371 Date: Mar. 29, 2023

Related U.S. Application Data
(60) Provisional application No. 63/086,915, filed on Oct. 2, 2020.

(51) Int. Cl. C07K 1/22; C07K 16/10; C07K 1/34; C07K 1/18; C07K 1/36

(52) U.S. Cl. CPC C07K 1/22; C07K 16/1003; C07K 1/34; C07K 1/18; C07K 1/36; C07K 2317/14

(57) ABSTRACT

The present disclosure relates to methods for reducing host cell protein content in a protein preparation recombinantly produced in a host cell in the manufacturing process of proteins intended for administration to a patient.

The present invention relates to the field of recombinant protein manufacturing. More particularly, the present invention provides a method for reducing host cell protein content in a protein preparation comprising a protein of interest recombinantly produced in a host cell in the manufacturing process of proteins intended for administration to a patient, such as therapeutic or diagnostic proteins.

Host Cell Proteins (HCPs) are proteins of the host cells that are involved in cell maintenance and growth, and protein synthesis and processing. However, in the realm of therapeutic or diagnostic proteins, the presence of HCPs threatens product quality and patient safety by posing concerns such as aggregation, product fragmentation by catalytic activity and/or immunogenicity. Hence, HCPs are identified as a critical quality attribute (CQA) of protein formulations. The formation of undesired aggregates and product fragmentation require additional purification steps to reduce/remove HCPs and these additional purification steps often result in reduced yield of the desired protein and increased overall manufacturing costs.

The challenges of eliminating HCPs from manufacturing processes, and attempts to improve the processes to reduce HCPs have been disclosed, for example as set forth in Gilgunn et al; Goey et al., Biotechnology Advances 36 (2018) 1223-1237; and Current Opinion in Chemical Engineering 2018, 22:98-106. However, these processes to remove HCPs have limitations. For example, in some instances, these disclosures demonstrate one or more of, incomplete removal of HCPs, inconsistency in processes in removal of HCPs leading to aggregation, co-purification of the desired proteins and HCPs, impaired product function, immunogenicity concerns in patients, and/or reduced pharmacokinetic properties such as half-life. Furthermore, the processes developed to remove HCPs often require for example, the need to work with increased volumes and additional purification steps, often resulting in increased manufacturing costs and reduced yield. In some instances, the applicability of the method is limited to a specific molecule and/or format. As such, there remains a need for alternative methods of reducing HCPs in the purification process of therapeutic or diagnostic proteins. Such alternative methods reduce HCPs preferably without affecting product stability, yield, or cost to ultimately maintain product quality and is amenable to large scale manufacturing and ensuring patient safety.

Accordingly, the present invention addresses one or more of the above problems by providing alternative methods of reducing HCPs in the preparation of therapeutic or diagnostic proteins. The methods of the present invention provide reproducible methods that are highly effective in removing HCPs, whilst preserving protein stability, reducing aggregation, maintaining product yield and has a potential to lower immunogenicity risk. Such methods can effectively remove HCPs without requiring increased protein preparation volume. Surprisingly, the methods of the present invention achieved HCP counts well below the industry acceptable standards of <100 ppm. Surprisingly, in embodiments the methods of the present invention achieved HCP counts of <50 ppm whilst preserving protein stability, reducing aggregation, and maintaining product yield. More surprisingly, other embodiments of the present invention achieved HCP counts of <20 ppm whilst preserving protein stability, reducing aggregation, and maintaining product yield. Furthermore, embodiments of the present invention provide methods of HCP removal that are applicable to a broad range of molecules. Other embodiments of the present invention enable the elimination of additional purification steps, resulting in a reduction in batch processing time, and decreased manufacturing costs.

Accordingly, particular embodiments, provide a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, raising the pH of the eluate to above about pH 6.0, subjecting the eluate to a depth filter, and obtaining a filtered protein preparation. In some embodiments the ionic strength of the eluate from the step of raising the pH to above about pH 6.0, is about 10 mM to about 45 mM. In some embodiments, the weak acid has no more than one pKa value less than 7.0, and the strong acid has no more than one pKa value less than 7.0. Preferably, the host cell protein content in the filtered protein preparation is reduced. More preferably, the host cell protein content in the filtered protein preparation is reduced to less than about 100 ppm, to less than about 50 ppm, or to less than about 20 ppm.

Accordingly, in particular embodiments, provided is a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, performing viral inactivation on the eluate, raising the pH of the eluate to above about pH 6.0, subjecting the eluate to a depth filter, and obtaining a filtered protein preparation. In some embodiments the ionic strength of the eluate from the step of raising the pH to above about pH 6.0, is about 10 mM to about 45 mM. In some embodiments, the weak acid has no more than one pKa value less than 7.0, and the strong acid has no more than one pKa value less than 7.0. Preferably, the host cell protein content in the filtered protein preparation is reduced. More preferably, the host cell protein content in the filtered protein preparation is reduced to less than about 100 ppm, to less than about 50 ppm, or to less than about 20 ppm.

Accordingly, in particular embodiments, provided is a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, performing viral inactivation comprising adjusting the pH of the eluate from said step of eluting the protein from the chromatography column, to below about pH 4.0, and wherein the eluate is maintained at below about pH 4.0 for about 0 minutes to about 180 minutes, raising the pH of the eluate to above about pH 6.0, subjecting the eluate comprising the protein to a depth filter, and obtaining a filtered protein preparation. In some embodiments the ionic strength of the eluate from the step of raising the pH to above about pH 6.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

Accordingly, in particular embodiments, provided is a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column, to below about pH 4.0, and wherein the eluate is maintained at below about pH 4.0 for about 0 minutes to about 180 minutes, raising the pH of the eluate to above about pH 6.0, subjecting the eluate comprising the protein to a depth filter, and obtaining a filtered protein preparation. In some embodiments the ionic strength of the eluate from the step of raising the pH to above about pH 6.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments of the invention, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid, wherein the concentration of the acetic acid is about 20 mM, and wherein the concentration of the phosphoric acid is about 5 mM to about 10 mM, adjusting the pH of the eluate comprising the protein from said step of eluting the protein from the chromatography column, to below about pH 4.0, and wherein the eluate is maintained at below about pH 4.0 for about 0 minutes about 180 minutes, raising the pH of the eluate to above about pH 6.0, subjecting the eluate comprising the protein to a depth filter, and obtaining a filtered protein preparation. In some embodiments the ionic strength of the eluate from the step of raising the pH to above about pH 6.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments of the invention, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is lactic acid, wherein the concentration of the acetic acid is about 20 mM, and wherein the concentration of the lactic acid is about 5 mM, adjusting the pH of the eluate comprising the protein from said step of eluting the protein from the chromatography column, to below about pH 4.0, and wherein the eluate is maintained at below about pH 4.0 for about 0 minutes to about 180 minutes, raising the pH of the eluate to above about pH 6.0, subjecting the eluate comprising the protein to a depth filter, and obtaining a filtered protein preparation. In some embodiments the ionic strength of the eluate from the step of raising the pH to above about pH 6.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column, wherein said step of adjusting the pH of the eluate comprises adding about 20 mM HCl to the eluate, wherein the pH of the eluate is adjusted to about pH 3.3 to about pH 3.7, and wherein the eluate is maintained at about pH 3.3 to about pH 3.7 for about 0 minutes to about 180 minutes, raising the pH of the eluate to above about pH 6.0, subjecting the eluate comprising the protein to a depth filter, and obtaining a filtered protein preparation. In some embodiments the ionic strength of the eluate from the step of raising the pH to above about pH 6.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column, wherein said step of adjusting the pH of the eluate comprises adding about 20 mM HCl to the eluate, wherein the pH of the eluate is adjusted to about pH 3.5, and wherein the eluate is maintained at about pH 3.5 for about 0 minutes to about 180 minutes, raising the pH of the eluate to above about pH 6.0, subjecting the eluate comprising the protein to a depth filter, and obtaining a filtered protein preparation. In some embodiments the ionic strength of the eluate from the step of raising the pH to above about pH 6.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some particular embodiments, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column to below about pH 4.0, and wherein the eluate is maintained at below about pH 4.0 for about 0 minutes to about 180 minutes, raising the pH of the eluate to about pH 6.5 to about pH 7.5 comprising adding about 250 mM Tris Buffer to the eluate, and subjecting the eluate comprising the protein to a depth filter, and obtaining a filtered protein preparation. In some embodiments, raising the pH of the eluate to about pH 6.5 to about pH 7.5 comprises adding about 100 mM to about 1000 mM Tris Buffer to the eluate. In some embodiments the ionic strength of the eluate from the step of raising the pH to above about pH 6.5 to about pH 7.5, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column to below about pH 4.0, and wherein the eluate is maintained at below about pH 4.0 for about 0 minutes to about 180 minutes, raising the pH of the eluate to about pH 7.0 comprising adding about 250 mM Tris buffer to the eluate, subjecting the eluate comprising the protein to a depth filter, and obtaining a filtered protein preparation. In some embodiments, raising the pH of the eluate to about pH 6.5 to about pH 7.5 comprises adding about 100 mM to about 1000 mM Tris Buffer to the eluate. In some embodiments the ionic strength of the eluate from the step of raising the pH to about pH 7.0, is about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column to below about pH 4.0, and wherein the eluate is maintained at below about pH 4.0 for about 0 minutes to about 180 minutes, raising the pH of the eluate to above pH about 6.0, subjecting the eluate comprising the protein to a depth filter, and obtaining a filtered protein preparation, wherein the eluate subjected to the depth filter has an ionic strength of about 10 mM to about 45 mM. Preferably, the host cell protein content in the filtered protein preparation is reduced. More preferably, the host cell protein content in the filtered protein preparation is reduced to less than about 100 ppm, to less than about 50 ppm, or to less than about 20 ppm.

In particular embodiments, the present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid is acetic acid and the strong acid is phosphoric acid or lactic acid, adjusting the pH of the eluate comprising the protein from said step of eluting the protein from the chromatography column to below about pH 4.0, and wherein the eluate is maintained at below about pH 4.0 for about 0 minutes to about 180 minutes and wherein viral inactivation is achieved.

The present disclosure provides a method of reducing host cell protein content in a protein preparation comprising a protein of interest recombinantly produced in a host cell comprising, subjecting the protein preparation recombinantly produced in a host cell to an affinity chromatography column, eluting the protein of interest from the chromatography column with a combination of acids comprising of a weak acid and a strong acid to obtain an eluate comprising the protein of interest, wherein the weak acid comprises acetic acid at a concentration of about 20 mM, and wherein the strong acid comprises of any one of phosphoric acid, formic acid, or lactic acid, and wherein the concentration of the strong acid is about 5 mM to about 10 mM, adjusting the pH of the eluate comprising the protein from said step of eluting the protein from the chromatography column, wherein said step of adjusting the pH of the eluate comprises adding any one of HCl, phosphoric acid, citric acid, or a combination of acetic acid plus phosphoric acid, to the eluate, wherein the pH is adjusted to below about pH 4.0, and wherein the eluate is maintained at below about pH 4.0 for about 0 minutes to about 180 minutes, raising the pH of the eluate to above about pH 6.0 to about pH 7.5, subjecting the eluate comprising the protein to a depth filter, and obtaining a filtered protein preparation. In some embodiments the ionic strength of the eluate from the step of raising the pH to above about pH 6.0 to about 7.5, is about 10 mM to about 45 mM. Preferably, the host cell protein content in the filtered protein preparation is reduced. More preferably, the host cell protein content in the filtered protein preparation is reduced to less than about 100 ppm. In further embodiments, the elution step comprises a combination of acids comprising of acetic acid and phosphoric acid, acetic acid and lactic acid, or acetic acid and formic acid, and wherein the step of adjusting the pH to below about pH 4.0 comprises adding any one of HCl, phosphoric acid, citric acid or a combination of acetic acid and phosphoric acid. In further embodiments, the elution step comprises of a combination of any one of about 20 mM acetic acid and about 10 mM phosphoric acid, about 20 mM acetic acid and about 5 mM phosphoric acid, or about 20 mM acetic acid and about 5 mM formic acid, and wherein the step of adjusting the pH to below about pH 4.0 comprises adding any one of about 20 mM HCl, about 15 mM to about 200 mM phosphoric acid, about 1000 mM citric acid, or a combination of about 20 mM acetic acid and about 10 mM phosphoric acid. In such embodiments the ionic strength of the eluate from the step of raising pH to above pH of about 6.0, is about 10 mM to about 45 mM.

In one aspect of the invention, the invention provides a method of reducing host cell protein content in a protein preparation comprising a protein of interest recombinantly produced in a host cell, comprising the steps of:
subjecting the protein preparation recombinantly produced in a host cell to an affinity chromatography column;
eluting the protein of interest from the chromatography column with a combination of acids comprising of a weak acid and a strong acid to obtain an eluate comprising the protein of interest; wherein the weak acid is acetic acid and the strong acid is phosphoric acid or lactic acid;
adjusting the pH of the eluate comprising the protein from said step of eluting the protein from the chromatography column, to below about pH 4.0, and wherein the eluate is maintained at below about pH 4.0 for about 0 minutes to about 180 minutes;
raising the pH of the eluate to above about pH 6.0;
subjecting the eluate comprising the protein to a depth filter, and
obtaining a filtered protein preparation.

Preferably, the host cell protein content in the filtered protein preparation is reduced. More preferably, the host cell protein content in the filtered protein preparation is reduced to less than about 100 ppm, to less than about 50 ppm, or to less than about 20 ppm.

In some embodiments, the protein is a therapeutic or diagnostic protein, e.g., an antibody, Fc Fusion protein, peptide, an immunoadhesin, an enzyme, a growth factor, a receptor, a hormone, a regulatory factor, a cytokine, an antigen, a peptide, or a binding agent. In some embodiments, the protein is an antibody, e.g., a monoclonal antibody, a chimeric antibody, a humanized antibody, a human antibody, a bispecific antibody, or an antibody fragment. In some embodiments, the protein is an IgG1 antibody or contains the Fc portion of an IgG1 antibody. In some embodiments, the protein is an anti-SARS-COV-2 antibody.

In another aspect of the invention, the invention provides a method of reducing host cell protein content in an anti-SARS-COV-2 antibody preparation recombinantly produced in a host cell comprising the steps of:
subjecting the anti-SARS-COV-2 antibody preparation recombinantly produced in a host cell to an affinity chromatography column, e.g., a Protein A affinity chromatography column;
eluting the anti-SARS-COV-2 antibody with a combination of acids comprising of acetic acid and phosphoric acid or a combination of acetic acid and lactic acid to obtain an eluate comprising the anti-SARS-COV-2 antibody;
adjusting the pH of the eluate comprising the anti-SARS-COV-2 antibody by addition of about 20 mM HCl, wherein the pH is adjusted to about pH 3.3 to about pH 3.7, and wherein the eluate is maintained at about pH 3.3 to about pH 3.7 for about 0 minutes to about 180 minutes;
raising the pH of the eluate comprising the anti-SARS-COV-2 antibody by addition of about 250 mM Tris Buffer, wherein the pH is raised to about pH 6.5 to about pH 7.5; and
subjecting the eluate comprising the anti-SARS-COV-2 antibody to a depth filter, and obtaining a filtered anti-SARS-COV-2 antibody preparation,
wherein host cell protein content in the filtered anti-SARS-COV-2 antibody preparation after depth filtration is reduced to about 0 ppm to about 100 ppm, and wherein the anti-SARS-COV-2 antibody is an IgG1 antibody.

In some embodiments of the invention, the present disclosure provides a method of reducing host cell protein content in an anti-SARS-COV-2 antibody preparation recombinantly produced in a host cell comprising, subjecting the anti-SARS-COV-2 antibody preparation recombinantly produced in a host cell to a Protein A chromatography column, eluting the anti-SARS-COV-2 antibody from the chromatography column with a combination of acids comprising of about 20 mM acetic acid and about 5 mM phosphoric acid, or a combination of acids comprising of about 20 mM acetic acid and about 10 mM phosphoric acid, or a combination of acids comprising of about 20 mM acetic acid and about 5 mM lactic acid to obtain an eluate comprising the anti-SARS-COV-2 antibody, adjusting the pH of the eluate comprising the anti-SARS-COV-2 antibody by addition of about 20 mM HCl, wherein the pH is lowered to about pH 3.3 to about pH 3.7, and wherein the eluate is maintained at about pH 3.3 to about pH 3.7 for about 0 minutes to about 180 minutes, raising the pH of the eluate comprising the anti-SARS-COV-2 antibody by addition of about 250 mM Tris Buffer, wherein the pH is raised to about pH 6.5 to about pH 7.5, subjecting the eluate comprising the anti-SARS-COV-2 antibody to a depth filter, and obtaining a filtered anti-SARS-COV-2 antibody preparation, wherein the host cell protein content in the filtered anti-SARS-COV-2 antibody preparation is about 0 ppm to about 100 ppm, and wherein the anti-SARS-COV-2 antibody is an IgG1 antibody. In some embodiments, raising the pH of the eluate to about pH 6.5 to about pH 7.5 comprises adding about 100 mM to about 1000 mM Tris Buffer to the eluate.

In some embodiments of the invention, the present disclosure provides a method of reducing host cell protein content in an anti-SARS-COV-2 antibody preparation recombinantly produced in a host cell comprising, subjecting the anti-SARS-COV-2 antibody preparation recombinantly produced in a host cell to a Protein A chromatography column, eluting the anti-SARS-COV-2 antibody from the chromatography column with a combination of acids comprising of about 20 mM acetic acid and about 5 mM phosphoric acid, or a combination of acids comprising of about 20 mM acetic acid and about 10 mM phosphoric acid, or a combination of acids comprising of about 20 mM acetic acid and about 5 mM lactic acid to obtain an eluate comprising the anti-SARS-COV-2 antibody, adjusting the pH of the eluate comprising the anti-SARS-COV-2 antibody with about 20 mM HCl, wherein the pH is adjusted to about pH 3.5, and wherein the eluate is maintained at about pH 3.5 for about 0 minutes to about 180 minutes, raising the pH of the eluate comprising the anti-SARS-COV-2 antibody with about 250 mM Tris Buffer, wherein the pH is raised to about pH 6.5 to about pH 7.5, subjecting the eluate comprising the anti-SARS-COV-2 antibody to a depth filter, and obtaining a filtered anti-SARS-COV-2 antibody preparation, wherein the host cell protein content in the filtered anti-SARS-COV-2 antibody preparation is about 0 ppm to about 100 ppm, and wherein the anti-SARS-COV-2 antibody is an IgG1 antibody. In some embodiments, raising the pH of the eluate to about pH 6.5 to about pH 7.5 comprises adding about 100 mM to about 1000 mM Tris Buffer to the eluate.

In some embodiments of the invention, the present disclosure provides a method of reducing host cell protein content in an anti-SARS-COV-2 antibody preparation recombinantly produced in a host cell comprising, subjecting the anti-SARS-COV-2 antibody preparation recombinantly produced in a host cell to a Protein A chromatography column, eluting the anti-SARS-COV-2 antibody from the chromatography column with a combination of acids comprising of about 20 mM acetic acid and about 5 mM phosphoric acid, or a combination of acids comprising of about 20 mM acetic acid and about 10 mM phosphoric acid, or a combination of acids comprising of about 20 mM acetic acid and about 5 mM lactic acid to obtain an eluate comprising the anti-SARS-COV-2 antibody, adjusting the pH of the eluate comprising the anti-SARS-COV-2 antibody by addition of about 20 mM HCl, wherein the pH is lowered to about pH 3.5, and wherein the eluate is maintained at about pH 3.5 for about 0 minutes to about 180 minutes, and wherein viral inactivation is achieved.

In some embodiments of the invention, the present disclosure provides a method of reducing host cell protein content in an anti-SARS-COV-2 antibody preparation recombinantly produced in a host cell comprising, subjecting the anti-SARS-COV-2 antibody preparation recombinantly produced in a host cell to a Protein A chromatography column, eluting the anti-SARS-COV-2 antibody from the chromatography column with a combination of acids comprising of about 20 mM acetic acid and about 5 mM phosphoric acid, or a combination of acids comprising of about 20 mM acetic acid and about 10 mM phosphoric acid, or a combination of acids comprising of about 20 mM acetic acid and about 5 mM lactic acid to obtain an eluate comprising the anti-SARS-COV-2 antibody, adjusting the pH of the eluate comprising the anti-SARS-COV-2 antibody by addition of about 20 mM HCl, wherein the pH is lowered to about pH 3.3 to about pH 3.7, and wherein the eluate is maintained at about pH 3.3 to about pH 3.7 for about 0 minutes to about 180 minutes, raising the pH of the eluate comprising the anti-SARS-COV-2 antibody with about 250 mM Tris Buffer, wherein the pH is raised to about pH 7.25, subjecting the eluate comprising the anti-SARS-COV-2 antibody to a depth filter, and obtaining a filtered anti-SARS-COV-2 antibody preparation, wherein the host cell protein content in the filtered anti-SARS-COV-2 antibody preparation is about 0 ppm to about 100 ppm, and wherein the anti-SARS-COV-2 antibody is an IgG1 antibody. In some embodiments, raising the pH of the eluate to about pH 7.25 comprises adding about 100 mM to about 1000 mM Tris Buffer to the eluate.

In some embodiments of the invention, the present disclosure provides a method of reducing host cell protein content in an anti-SARS-COV-2 antibody preparation recombinantly produced in a host cell comprising, subjecting the anti-SARS-COV-2 antibody preparation recombinantly produced in a host cell to a Protein A chromatography column, eluting the anti-SARS-COV-2 antibody from the chromatography column with a combination of acids comprising of about 20 mM acetic acid and about 5 mM phosphoric acid, or a combination of acids comprising of about 20 mM acetic acid and about 5 mM phosphoric acid, or a combination of acids comprising of about 20 mM acetic acid and about 5 mM lactic acid to obtain an eluate comprising the anti-SARS-COV-2 antibody, adjusting the pH of the eluate comprising the anti-SARS-COV-2 antibody by addition of about 20 mM HCl, wherein the pH is lowered to about pH 3.5, and wherein the eluate is maintained at about pH 3.5 for about 0 minutes to about 180 minutes, raising the pH of the eluate comprising the anti-SARS-COV-2 antibody by addition of about 250 mM Tris Buffer, wherein the pH is raised to about pH 7.25, subjecting the eluate comprising the anti-SARS-COV-2 antibody to a depth filter, and obtaining a filtered anti-SARS-COV-2 antibody preparation, wherein the host cell protein content in the filtered anti-SARS-COV-2 antibody preparation is about 0 ppm to about 100 ppm, and wherein the anti-SARS-COV-2 antibody is an IgG1 antibody. In some embodiments, raising the pH of the eluate to about pH 7.25 comprises adding about 100 mM to about 1000 mM Tris Buffer to the eluate.

In some embodiments, the invention provides methods of reducing host cell protein content in an anti-SARS-COV-2 antibody preparation recombinantly produced in a host cell,

In some embodiments, the anti-SARS-COV-2 antibody is bamlanivimab. In some embodiments, the anti-SARS-COV-2 antibody comprises a variable heavy chain comprising of an amino acid sequence of SEQ ID NO: 1 and a variable light chain comprising of an amino acid sequence of SEQ ID NO: 2. In some embodiments, the anti-SARS-COV-2 antibody comprises a heavy chain comprising of an amino acid sequence of SEQ ID NO: 3 and a light chain comprising of an amino acid sequence of SEQ ID NO: 4. In other embodiments, the anti-SARS-COV-2 antibody is etesevimab. In yet other embodiments, the anti-SARS-COV-2 antibody comprises a variable heavy chain comprising of an amino acid sequence of SEQ ID NO: 5 and a variable light chain comprising of an amino acid sequence of SEQ ID NO: 6. In yet further embodiments, the anti-SARS-COV-2 antibody comprises a heavy chain comprising of an amino acid sequence of SEQ ID NO: 7 and a light chain comprising of an amino acid sequence of SEQ ID NO: 8. In some embodiments, the anti-SARS-COV-2 antibody is bebtelovimab. In yet other embodiments, the anti-SARS-COV-2 antibody comprises a variable heavy chain comprising of an amino acid sequence of SEQ ID NO: 9 and a variable light chain comprising of an amino acid sequence of SEQ ID NO: 10. In yet further embodiments, the anti-SARS-COV-2 antibody comprises a heavy chain comprising of an amino acid sequence of SEQ ID NO: 11 and a light chain comprising of an amino acid sequence of SEQ ID NO: 12.

In some embodiments, the protein, e.g., therapeutic or diagnostic protein, is produced in mammalian cells. In some embodiments, the mammalian cell is a Chinese Hamster Ovary (CHO) cells, or baby hamster kidney (BHK) cells, murine hybridoma cells, or murine myeloma cells. In some embodiments, the therapeutic or diagnostic protein is produced in bacterial cells. In other embodiments, the therapeutic or diagnostic protein is produced in yeast cells.

In some embodiments, the invention provides methods wherein the method of reducing host cell protein content in a protein preparation comprising a protein of interest recombinantly produced in a host cell after subjecting to a depth filter is further subjected to ion exchange chromatography.

In some embodiments, the present disclosure provides a method of reducing host cell protein content in a protein preparation comprising a protein of interest recombinantly produced in a host cell, wherein the host cell protein content in the protein preparation is reduced to less than about 100 ppm. In other embodiments the host cell protein content in the protein preparation is reduced to less than about 50 ppm. In other embodiments the host cell protein content in the protein preparation is reduced to less than about 20 ppm. In other embodiments the host cell protein content in the protein preparation is reduced to less than about 10 ppm. In other embodiments the host cell protein content in the protein preparation is reduced to about 0 ppm.

In some embodiments, the present disclosure provides a method of reducing host cell protein content in a protein preparation comprising a protein of interest recombinantly produced in a host cell, wherein the host cell protein content in the protein preparation comprises PLBL2, and wherein the PLBL2 is reduced to less than about 100 ppm. In other embodiments the PLBL2 is reduced to less than about 50 ppm. In other embodiments the PLBL2 is reduced to less than about 20 ppm. In other embodiments the PLBL2 is reduced to less than about 10 ppm. In other embodiments the PLBL2 is reduced to about 0 ppm.

In some embodiments, the present disclosure provides a method of reducing host cell protein content in a protein preparation comprising a protein of interest recombinantly produced in a host cell, wherein the host cell protein content in the protein preparation is reduced by about 97% after depth filtration from protein capture eluate. In other embodiments the host cell protein content in the protein preparation is reduced by about 99%. In other embodiments the host cell protein content in the protein preparation is reduced by about 99.9%. In other embodiments the host cell protein content in the protein preparation is reduced by about 99.99%. In other embodiments the host cell protein content in the protein preparation is reduced by about 100%.

In some embodiments, the present disclosure provides a method of reducing host cell protein content in a protein preparation comprising a protein of interest recombinantly produced in a host cell, wherein the host cell protein content in the protein preparation comprises PLBL2, and wherein the PLBL2 is reduced to less than about 100 ppm. In other embodiments the PLBL2 is reduced to less than about 50 ppm. In other embodiments the PLBL2 is reduced to less than about 20 ppm. In other embodiments the PLBL2 is reduced to less than about 10 ppm. In other embodiments the PLBL2 is reduced to about 0 ppm.

In some embodiments the present invention provides methods of reducing host cell protein content in a protein preparation comprising a protein of interest recombinantly produced in a host cell, wherein the protein preparation is subjected to depth filtration. In some embodiments the depth filter is one or more of X0SP, C0SP, X0HC, Emphaze™ AEX Hybrid Purifier, Zeta Plus (ZB Media) such as, Zeta Plus (60ZB05A), Zeta Plus (90ZB05A), or Zeta Plus (90ZB08A).

In some embodiments the present disclosure provides a method of reducing host cell protein content in a protein preparation comprising a protein of interest recombinantly produced in a host cell, wherein the ionic strength of the eluate from the step of raising pH to above pH of about 6.0, is about 10 mM to about 45 mM. In some embodiments, the ionic strength is less than about 30 mM. In some embodiments, the ionic strength is less than about 20 mM. In other embodiments the ionic strength is less than about 15 mM.

In some embodiments the invention provides methods wherein the protein preparation comprising a protein of interest recombinantly produced in a host cell is subjected to a chromatography column. In some embodiments, the chromatography column is one or more of an affinity column, an ion exchange column, a hydrophobic interaction column, a hydroxyapatite column, or a mixed mode column. In some embodiments, the affinity chromatography column is a Protein A column, a Protein G column, or a Protein L column. In other embodiments, the ion exchange chromatography column is an anion exchange column or a cation exchange column. In some embodiments, the invention provides methods wherein the HCPs are sufficiently removed from the final product.

In some embodiments, the invention provides methods of reducing host cell protein content in a protein preparation comprising a protein of interest recombinantly produced in a host cell, wherein the protein is a therapeutic or diagnostic protein. In further embodiments the therapeutic or diagnostic protein is an antibody, an Fc fusion protein, an immunoadhesin, an enzyme, a growth factor, a receptor, a hormone, a regulatory factor, a cytokine, an antigen, or a binding agent. In further embodiments, the antibody is a monoclonal antibody, a chimeric antibody, a humanized antibody, a human antibody, a bispecific antibody, or an antibody fragment.

In another aspect, provided herein are pharmaceutical compositions comprising the protein preparation, nucleic acid, or vector described herein. In further aspects the present disclosure provides a composition produced by the methods as described herein. In yet other embodiments the present disclosure provides a composition produced by the methods as described herein, wherein the host cell protein content in the composition is less than about 100 ppm.

The term “Host cell proteins” (HCPs) are proteins of the host cells that are involved in cell maintenance and growth, and protein synthesis and processing. Such HCPs for example include those from Chinese Hamster Ovary (CHO) cells, e.g., Phospholipase B-like 2 (PLBL2), vLPL (lipoprotein lipase), vLAL (lysosomal acid lipase, lysosomal lipase, LIPA), vPLA2 (phospholipase A2), vPPT1 (palmitoyl-protein thioesterase 1), PLBD2, and/ or Peroxiredoxin.

The term “weak acid” refers to an acid with a lowest pKa of >˜4. Examples of weak acids include but are not limited to, acetic acid, succinic acid, and 2-(N-morpholino)ethanesulfonic acid.

The term “strong acid” refers to an acid with a lowest pKa of <˜4. Examples of strong acids include but are not limited to, phosphoric acid, lactic acid, formic acid, malic acid, malonic acid, glycolic acid, citric acid, tartaric acid, and hydrochloric acid.

The term “depth filter” refers to a filter element that uses a porous filtration medium which retains particles throughout the medium (within and on the medium) rather than just on the surface of the medium. Depth filters may additionally have adsorptive capabilities resulting from the chemical properties of the materials from which they are composed. Examples of commercially available depth filters include, but are not limited to X0SP, C0SP, X0HC, Emphaz™ AEX Hybrid Purifier, Zeta Plus (60ZB05A), Zeta Plus (90ZB05A), and Zeta Plus (90ZB08A). The term “depth filtration” refers to the act of passing a liquid material which may be heterogeneous or homogeneous through a depth filter. In some embodiments, the liquid material comprises a protein preparation comprising a protein of interest.

The term “ionic strength,” when referring to a solution, is a measure of concentration of ions in that solution. Ionic strength (I) is a function of ion concentration, cᵢ, and net charge, zᵢ, for all ionic species. To determine ionic strength, Formula 1 is used.

$$I = \frac{1}{2} {\sum }_{i} {c}_{i} {z}_{i}^{2} \qquad ( 1 )$$

A “protein preparation” is the material or solution provided for a purification process or method which contains a therapeutic or diagnostic protein of interest and which may also contain various impurities. Non-limiting examples may include, for example, harvested cell culture fluid (HCCF), harvested cell culture material, clarified cell culture fluid, clarified cell culture material, the capture pool, the recovered pool, and/or the collected pool containing the therapeutic or diagnostic protein of interest after one or more centrifugation steps, and/or filtration steps, the capture pool, the recovered protein pool and/or the collected pool containing the therapeutic or diagnostic protein of interest after one or more purification steps.

The term “impurities” refers to materials that are different from the desired protein product. The impurity includes, without limitation: host cell materials, such as host cell proteins, CHOP; leached Protein A; nucleic acid; a variant, size variant, fragment, aggregate, or derivative of the desired protein; another protein; endotoxin; viral contaminant; cell culture media component, etc.

The terms “protein” and “polypeptide” are used interchangeably herein to refer to a polymer of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, proteins containing one or more analogs of an amino acid (including, for example, unnatural amino acids, etc.), as well as other modifications known in the art. Examples of proteins include, but are not limited to, antibodies, peptides, enzymes, receptors, hormones, regulatory factors, antigens, binding agents, cytokines, Fc fusion proteins, immunoadhesin molecules, etc.

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4). The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)₂, Fv fragments, scFv antibody fragments, scFab, disulfide-linked Fvs (sdFv), a Fd fragment.

The term “anti-SARS-CoV2 antibody” as used herein refers to an antibody that binds the spike (S) protein of SARS-CoV-2. The amino acid sequence of SARS-CoV-2 spike (S) protein has been described before, for example, GenBank Accession No: YP_009724390.1.

The term “ultrafiltration” or “filtration” is a form of membrane filtration in which hydrostatic pressure forces a liquid against a semipermeable membrane. Suspended solids and solutes of high molecular weight are retained, while water and low molecular weight solutes pass through the membrane. In some examples, ultrafiltration membranes have pore sizes in the range of 1 μm to 100 μm. The terms “ultrafiltration membrane” “ultrafiltration filter” “filtration membrane” and “filtration filter” may be used interchangeably. Examples of filtration membranes include but are not limited to polyvinylidene difluoride (PVDF) membrane, cellulose acetate, cellulose nitrate, polytetrafluoroethylene (PTFE, Teflon), polyvinyl chloride, polyethersulfone, glass fiber, or other filter materials suitable for use in a cGMP manufacturing environment.

As used herein, numeric ranges are inclusive of the numbers defining the range.

The term “EU numbering”, which is recognized in the art, refers to a system of numbering amino acid residues of immunoglobulin molecules. EU numbering is described, for example, at Kabat et al., Sequences of Proteins of Immunological Interest, 5th Ed. Public Health Service, National Institutes of Health, Bethesda, MD. (1991); Edelman, G. M, et al., Proc. Natl. Acad. USA, 63, 78-85 (1969); and http://www.imgt.org/IMGTScientificChart/Numbering/Hu_IGHGnber.html#refs. The term “Kabat numbering” is recognized in the art as referring to a system of numbering amino acid residues which are more variable (i.e., hypervariable) than other amino acid residues in heavy and light chain variable regions (see, for example, Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)). The term “North numbering”, refers to a system of numbering amino acid residues which are more variable (i.e., hypervariable) than other amino acid residues in heavy and light chain variable regions and is based, at least in part, on affinity propagation clustering with a large number of crystal structures, as described in (North et al., A New Clustering of Antibody CDR Loop Conformations, Journal of Molecular Biology, 406:228-256 (2011).

As used herein, the term “affinity chromatography” refers to a chromatographic method for separating biochemical mixtures (e.g., a protein and undesired biomolecule species) based on specific, reversible interactions between biomolecules. Exemplary embodiments of affinity chromatography include Protein A affinity, Protein G affinity, Protein L affinity, kappa affinity ligand chromatography (such as CaptureSelect™, KappaXL™, KappaSelect™, KappaXP™) or lambda affinity ligand chromatography.

A protein of the present disclosure can be incorporated into a pharmaceutical composition which can be prepared by methods well known in the art and which comprise a protein of the present disclosure and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with the protein, retains the molecule's activity and is non-reactive with the patient's immune system.

Expression vectors capable of directing expression of genes to which they are operably linked are well known in the art. Expression vectors can encode a signal peptide that facilitates secretion of the polypeptide(s) from a host cell. The signal peptide can be an immunoglobulin signal peptide or a heterologous signal peptide. Each of the expressed polypeptides may be expressed independently from different promoters to which they are operably linked in one vector or, alternatively, may be expressed independently from different promoters to which they are operably linked in multiple vectors. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

A host cell refers to cells stably or transiently transfected, transformed, transduced or infected with one or more expression vectors expressing one or more protein of the present disclosure. Creation and isolation of host cell lines producing proteins of the present disclosure can be accomplished using standard techniques known in the art. Mammalian cells are preferred host cells for expression of proteins of the present disclosure. Particular mammalian cells include HEK 293, NS0, DG-44, and CHO. Preferably, the proteins are secreted into the medium in which the host cells are cultured, from which the proteins can be recovered or purified by for example using conventional techniques. For example, the medium may be applied to and eluted from a Protein A affinity chromatography column and/or a kappa affinity ligand or lambda affinity ligand chromatography column. Undesired biomolecule species including soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, NY (1994).

### EXAMPLES

Host cell protein (HCP) measurements by LCMS: to assess purification impact on host cell protein (HCP) levels in the examples which follow, samples are analyzed by peptide mapping/LC-MS/MS HCP profiling via, e.g., a Ultra Performance Liquid Chromatography (UPLC) coupled to a Thermo Scientific mass spectrometer. In this analysis, the samples are subjected to digestion by trypsin, reduced/precipitated with dithiothreitol (DTT), followed by transfer and acidification of the supernatant in a HPLC vial for LC-MS/MS analysis. The LC-MS/MS data is analyzed by Proteome Discoverer against CHO-K1 protein database with added antibody, spike, and control protein sequences. The HCP content is reported as total parts per million (ppm) of HCP per sample for total HCP content (e.g., ng of HCP per mg of product). Additionally, phospholipase B-like 2 (PLBL2) content is also provided.

HCP measurements by ELISA: HCP content in the samples is also assessed in the examples which follow by an ELISA assay using a Gyrolab® CHO-HCP Kit 1 (Cygnus Technologies, performed per manufacturer instructions). The HCP content is reported as total parts per million (ppm) of HCP per sample for total HCP content.

### Example 1—HCP reduction in mAb1 (etesevimab) Purification Process

Protein Capture step: A sanitized Protein A column (Mab Select SuRe Protein A media) is equilibrated and mAb1 (etesevimab) cell-free bioreactor harvest is loaded onto the Protein A column and three washes of the Protein A column are performed using 20 mM Tris (pH 7.0) as the last wash. mAb1 is eluted from the column using 5 column volumes (CVs) of 20 mM acetic acid +5 mM phosphoric acid. The main product fraction is collected into a single bulk fraction by using absorbance-based peak cutting on the frontside and backside.

Low pH Viral Inactivation Step and Neutralization Step: Viral inactivation is conducted by adjusting the pH of the collected main product fraction (protein capture eluate bulk fraction) containing mAb1 to a pH between 3.30 and 3.60 by the addition of 20 mM HCl. The mixture is incubated at 18° C. to 25° C. for about 180 min. The mixture is then neutralized to a pH of 7.0 using 250 mM Tris base pH unadjusted buffer.

Depth Filtration Step: A depth filter (X0SP, Millipore) is flushed with water for injection (WFI). The mAb1 mixture, obtained from the low pH viral inactivation step and neutralization step, is applied to the depth filter with a loading of 1200 g/m² (grams of mAb per m² of depth filter membrane area). The loaded depth filter is flushed with WFI. The filtrate from the depth filter, optionally inclusive of the post-loading WFI flush, is neutralized to pH 8.0 using 250 mM Tris base pH unadjusted buffer.

Anion Exchange (AEX) Chromatography Step: A sanitized column (Q Sepharose Fast Flow Anion Exchange Chromatography Media, or QFF) is equilibrated with 2 CVs of 20 mM Tris (pH 8.0). The mAb1 solution, obtained from the depth filtration step, is loaded onto the column at a loading of 25 g to 100 g per liter of resin, and an additional wash is performed with the equilibration buffer. mAb1 is collected by absorbance-based peak cutting on the frontside and backside of the peak area formed by the unbound fraction plus the additional wash.

Results: Using the purification process described, the total HCP level as measured by LC-MS is:
23299 ppm after Protein A elution;
13 ppm after X0SP depth filtration;
2 ppm after AEX chromatography.

Depth filter Set 1 assessment for mAb1: mAb1 is processed through Protein A, low pH viral inactivation, neutralization, and depth filtration steps essentially as described above. Four different depth filters: Emphaze™ AEX Hybrid Purifier, Zeta Plus BC25-60ZB05A, Zeta Plus BC25-90ZB05A, and Zeta Plus BC25-90ZB08A (3M) are tested at a loading of 2000 g/m² as shown in Table 1. The results in Table 1 show a significant reduction in total HCP content after depth filtration by LCMS (ranging from 24 to 31 ppm) and/or ELISA (ranging from 6 to 16 ppm) for the 4 depth filters tested when compared to the total HCP content observed after Protein A elution by LCMS (28901 ppm) and Elisa (527 ppm).

TABLE 1
mAb1 total HCP content before and after depth filtration

| Total HCP content after | | | Total HCP content after | |
|---|---|---|---|---|
| Protein A elution (ppm) | | | depth filtration (ppm) | |
| LCMS | ELISA | Depth filter | LCMS | ELISA |
| 28901 | 527 | Emphaze ™ AEX | not available | 16 |
| | | Hybrid Purifier | | |
| | | Zeta Plus BC25 - | 31 | 8 |
| | | (60ZB05A) | | |
| | | Zeta Plus BC25 - | 29 | 7 |
| | | (90ZB05A) | | |
| | | Zeta Plus BC25 - | 24 | 6 |
| | | (90ZB08A) | | |

### Example 2—HCP Reduction in mAb2 (bamlanivimab) Purification Process

Protein A elution buffer comparison: mAb2 (bamlanivimab) is prepared essentially as described for mAb1 in Example 1 with the following exceptions: 1) mAb 2 is eluted from the Protein A capture column using the buffer combinations as listed in Table 2, 2) after the low pH viral inactivation step and before the depth filtration step, the mAb2 solution is neutralized to a pH of 7.25 instead of 7.0 using 250 mM Tris base pH unadjusted buffer, and 3) the AEX chromatography is performed using Poros XQ resin. HCP content (both total HCP content and PLBL2 content) is assessed via LCMS, after purification unit operations as listed in Tables 2 and 3.

The results in Tables 2 and 3, show that the total HCP and PLBL2 content after the depth filtration step was reduced for all 3 acid combinations tested. Specifically, the combinations of 20 mM acetic acid +5 mM phosphoric acid and 20 mM acetic acid +5 mM L-lactic acid showed a greater reduction of total HCP content to less than 20 ppm after depth filtration when compared to the 20 mM acetic acid +5 mM citric acid combination. Furthermore, the PLBL2 content after the depth filtration step with the 20 mM acetic acid +5 mM phosphoric acid and 20 mM acetic acid +5 mM L-lactic acid combinations was reduced to below limit of quantification.

TABLE 2
mAb2 total HCP content using different Protein A elution buffers

| | | Total HCP | |
|---|---|---|---|
| | Total HCP | by LCMS | Total HCP by |
| | by LCMS | detection | LCMS |
| | detection | after | detection |
| | after | X0SP depth | after AEX |
| Protein A elution | Protein A | filtration | chromatography |
| buffer | elution (ppm) | (ppm) | (ppm) |
| 20 mM acetic acid + | 71022 | 469 | 55 |
| 5 mM citric acid | | | |
| 20 mM acetic acid + | 77892 | 7 | 11 |
| 5 mM phosphoric acid | | | |
| 20 mM acetic acid + | 78669 | 16 | Below limit of |
| 5 mM L-lactic acid | | | quantitation |

TABLE 3
mAb2 PLBL2 content using different Protein A elution buffers

| | PLBL2 | PLBL2 | PLBL2 |
|---|---|---|---|
| | by LCMS | by LCMS | by LCMS |
| | detection | detection | detection |
| | after | after | after |
| | Protein A | X0SP depth | AEX |
| Protein A elution | elution | filtration | chromatography |
| buffer | (ppm) | (ppm) | (ppm) |
| 20 mM acetic acid + | 356 | 454 | 8 |
| 5 mM citric acid | | | |
| 20 mM acetic acid + | 351 | Below limit of | Below limit of |
| 5 mM phosphoric acid | | quantitation | quantitation |
| 20 mM acetic acid + | 404 | Below limit of | Below limit of |
| 5 mM L-lactic acid | | quantitation | quantitation |

Depth filter set 2 assessment: mAb 2 is prepared essentially as described for mAb1 with the following exceptions: 1) after the low pH viral inactivation step and before the depth filtration step, the mAb2 solution is neutralized to, a pH of 7.25 instead of 7.0 using 250 mM Tris base pH unadjusted buffer, and 2) the depth filtration step is performed with the depth filters shown in Table 4.

The results in Table 4 show that the total HCP and PLBL2 content after depth filtration with all 3 set 2 depth filters (X0SP, C0SP, X0HC, (Millipore)) loaded of 1500 g/m² was reduced to less than 20 ppm after the depth filtration step.

TABLE 4
mAb2 HCP total and PLBL2 content
before and after depth filtration

| Total HCP | PLBL2 | | | |
|---|---|---|---|---|
| content by | content by | | Total HCP | PLBL2 content |
| LCMS | LCMS | | content | by LCMS |
| after | after | | by LCMS | after |
| Protein A | Protein A | | after depth | depth |
| elution | elution | Depth | filtration | filtration |
| (ppm) | (ppm) | filter | (ppm) | (ppm) |
| 74528 | 543 | X0SP | 3 | Below limit of |
| | | | | quantitation |
| | | C0SP | 18 | 5 |
| | | X0HC | 2 | Below limit of |
| | | | | quantitation |

### Example 3. HCP Reduction in mAb3 (bebtelovimab) Purification Process

mAb3 (bebtelovimab) is prepared using the protein capture, low pH viral inactivation, neutralization, and depth filtration steps essentially as described for mAb1 in Example 1, except using a X0SP depth filter with a loading of 900 g/m². Using the described purification process the total HCP level as measured by LCMS is:
179964 ppm after the Protein A elution,
77 ppm after X0SP (Millipore) depth filtration.

### Example 4. HCP Reduction in Bispecific Antibody (mAb4) Purification Process

A bispecific antibody mAb4 is prepared using the protein capture step essentially as described for mAb 1 in Example 1, except using a Protein L affinity capture column (Cytiva) and eluting with the buffer systems shown in Table 5. The total HCP content is measured by ELISA giving a range of about 1300 to about 2500 ppm. Following protein capture, low pH viral inactivation is performed essentially as described for mAb 1 in 10 Example 1, except using the titrants listed in Table 5, followed by neutralization up to pH 7.0 using 500 mM Tris base pH unadjusted buffer. The depth filtration step is performed essentially as described for mAb1 in Example 1 using a X0SP depth filter at a loading of 1200 g/m². The HCP content is measured after depth filtration by ELISA.

The results in Table 5, show significant reduction in total HCP content to less than ≤50 ppm for Entries 1 to 7 following depth filtration, where the ionic strength of the mixtures applied to the depth filter was less than about 45 mM. In addition, a correlation between the ionic strength of the mixtures applied to the depth filter and the total HCP content after the depth filtration was observed. Furthermore, Entry 2 shows that although ionic strength can be decreased by diluting the buffer, providing low HCP content after depth filtration, however the volume increase from dilution can be disadvantageous to manufacturing processes.

TABLE 5
HCP levels in mAb4 preparations following
Protein L elution and depth filtration

| | | | Ionic strength | Total HCP |
|---|---|---|---|---|
| | | | of mixture | content by |
| | | Low pH viral | applied to | ELISA after |
| | Protein L | inactivation | depth filter | X0SP depth |
| Entry | elution buffer | titrant | (mM) | filtration (ppm) |
| 1 | 20 mM acetic | 20 mM acetic | 38 | 38 |
| | acid + 10 mM | acid + 10 mM | | |
| | phosphoric acid | phosphoric acid | | |
| 2 | 20 mM acetic | 20 mM acetic | 13 | 18 |
| | acid + 10 mM | acid + 10 mM | (after 1:2 H₂O | |
| | phosphoric acid | phosphoric acid | dilution)* | |
| 3 | 20 mM acetic | 20 mM HCl | 36 | 35 |
| | acid + 10 mM | | | |
| | phosphoric acid | | | |
| 4 | 20 mM acetic | 20 mM HCl | 27 | 30 |
| | acid + 5 mM | | | |
| | phosphoric acid | | | |
| 5 | 20 mM acetic | 20 mM HCl | 23 | 26 |
| | acid + 5 mM | | | |
| | formic acid | | | |
| 6 | 20 mM acetic | 200 mM | 43 | 50 |
| | acid + 10 mM | phosphoric acid | | |
| | phosphoric acid | | | |
| 7 | 20 mM acetic | 15 mM | 37 | 36 |
| | acid + 10 mM | phosphoric acid | | |
| | phosphoric acid | | | |
| 8 | 20 mM acetic | 1000 mM | 64 | 209 |
| | acid + 10 mM | citric acid | | |
| | phosphoric acid | | | |

*following low pH viral inactivation and neutralization to pH 7.0 with 500 mM Tris, the mAb4 solution is diluted with 2 parts water (1:2 ratio of mAb4 solution:H₂O)

### Example 5. HCP Reduction in mAb5 Purification Processes

mAb5 is prepared using the protein capture step essentially as described for mAb1 in Example 1, except the elution step is performed with the buffer systems shown in Table 6. The total HCP content is measured by ELISA giving a range of about 2800 to about 3200 ppm. Following protein capture, the low pH viral inactivation step is performed essentially as described for mAb1 in Example 1, followed by a neutralization step at either pH 5.0 or pH 7.0 using 500 mM Tris base pH unadjusted buffer. The depth filtration step is performed essentially as described for mAb1 in Example 1 using a X0SP depth filter at a loading of 1000 g/m². The HCP content after the depth filtration step is measured by ELISA.

The results in Table 6 show a significant reduction in total HCP content to less than ≤50 ppm for mAb5 following depth filtration when the pH of the mixture applied to the depth filter is pH 7.0. Total HCP content is reduced to a lesser extent when the pH of the mixture applied to the depth filter is pH 5.0.

TABLE 6
HCP levels in mAb5 preparations following
Protein A elution and depth filtration

| | | pH of material | HCP content |
|---|---|---|---|
| | Protein A elution | applied to | after depth |
| Antibody | buffer | depth filter | filtration |
| mAb5 | 20 mM acetic acid + 5 mM | pH 5 | 338 |
| | lactic acid | pH 7 | 41 |
| | 20 mM acetic acid + 5 mM | pH 5 | 331 |
| | phosphoric acid | pH 7 | 9 |

### Example 6. Method for Determination of Ionic Strength During Biomolecule Purification Processes

A method for the estimation of ionic strength based on what is known of the buffer compositions during biomolecule purification unit processes is herein described. The ionic strength (I) of a solution is a measure of concentration of ions in that solution, and is a function of species concentration, cᵢ, and net charge, zᵢ, for all ionic species. To determine ionic strength, Formula 1 is used.

$$I = \frac{1}{2} {\sum }_{i} {c}_{i} {z}_{i}^{2} \qquad ( 1 )$$

Strong electrolytes: for strong electrolytes at low concentrations (e.g., below 50 mM), complete dissociation is assumed. With complete dissociation, the composition is easily calculated making ionic strength calculations straightforward. For example, a solution of 50 mM NaCl dissociates to give 50 mM each of Na⁺ and Cl⁻ with an ionic strength of 0.5×[50 mM×1²+50 mM×(−1)²]=50 mM. As another example, 50 mM Na₂SO₄ dissociates to give 100 mM of Na⁺ and 50 mM of SO₄²⁻, giving an ionic strength of 0.5×[φmM×1²+50 mM×(−2)₂]=150 mM. With no buffering species, near-neutral pH is expected in these calculations such that concentrations of ions from the dissociation of water do not contribute meaningfully to the ionic strength. The dissociation constant of water is taken to be K_{w}=[H⁺][OH⁻]10⁻¹⁴ with [H⁺]=10^{-pH} where the square brackets indicate concentrations. For the purpose of calculations herein, physical interpretation of H⁺ ions (as opposed to hydronium ions, for example) is not necessary, and likewise it is not necessary to distinguish between H⁺ concentration and activity. Buffered systems: for buffered systems complete dissociation cannot be assumed. Acid dissociation constants of the buffers must be used to determine the proportion of the buffer in the acid and base forms. For a generic acid, HA, that dissociates H⁺ and A⁻ Formula 2 relates to the acid dissociation constant, Kₐ, and the species concentrations:

$${K}_{a} = \frac{[ {H}^{+} ] [ {A}^{-} ]}{[ H A ]} \qquad ( 2 )$$

The acid dissociation constant is often used in the logarithmic form of pKₐ=−log₁₀(Kₐ). The thermodynamic pKₐ, denoted as pK_{a,0}, is available in the literature for many buffers of interest. However, the effective pKₐ of a buffer diverges from the thermodynamic value except in very dilute solution due to deviation of activity coefficients from unity. For moderately dilute solutions considered in this disclosure, the extended Debye Hückel equation or Davies equation were used to account for non-unity activity coefficients. Values for some of the constants found in literature may differ slightly but give similar results in the range of ionic strength values of interest in the present disclosure. The extended Debye Hückel equation is provided as Formula 3:

$$p {K}_{a} = p {K}_{a , 0} + \frac{0.51 n \sqrt{I}}{1 + 1.6 \sqrt{I}} \qquad ( 3 )$$

The Davies equation is provided as Formula 4:

$$p {K}_{a} = p {K}_{a , 0} + 0.51 n ( \frac{\sqrt{I}}{1 + \sqrt{I}} - 0.3 \sqrt{I} ) \qquad ( 4 )$$

where n=2z−1 and z is the net charge of the acidic buffer form for calculating n (Scopes, Protein Purification: Principles and Practices, 2013).

Since pKₐ is a function of ionic strength, the composition and ionic strength cannot be determined independently, but are part of a system of equations. The system of equations includes the aforementioned equations for ionic strength, acid dissociation constants for each buffer, and pKₐ equations for each buffer, and also includes an electroneutrality condition and a total species balance for each buffer. With this system of equations, several values may be estimated. For example, a known solution pH can be used to estimate an acid-based ratio for a buffer formulation, or conversely an acid-based ratio can be used to estimate a solution pH and corresponding titration volumes. In any of these applications, the ionic strength can be estimated, to help guide rational selection of eluent and titrant options.

To calculate the ionic strength relevant to the buffered systems in the present disclosure, such as that of the feed material for depth filtration, the buffer composition of the solution is needed. This composition can be reasonably estimated based on the volumes and compositions of the buffers and titrants used in the process. Ion measurement techniques known in the field may also be used to estimate the composition.

As a starting point for estimating the solution composition, one possible methodology is to assume that the affinity column eluate pool has a buffer composition identical to that of the eluent with the exception of being buffered at the measured pH of the eluate pool. For example, if the protein of interest is eluted from a Protein A column with 20 mM acetic acid, 5 mM lactic acid and the eluate pool has a measured pH of 4.2, the assumption would be made that the buffer composition of the eluate pool is 20 mM acetate, 5 mM lactate, and sufficient NaOH to bring pH to 4.2; this would equate to about ˜8.2 mM NaOH. Because only the total sodium cation, Na⁺, content is important to the calculation, it does not matter whether the eluate sodium content is assumed to originate from sodium acetate, sodium phosphate, sodium hydroxide, or any combination thereof, so the convention of attributing the sodium to NaOH is used for convenience.

Having used the eluent composition and eluate pH to estimate the buffer composition of the eluate, the solution titrations are then considered. For example, with an estimated eluate composition of 20 mM acetate, 5 mM lactate, ˜8.2 mM NaOH at pH 4.2, if the volume of 20 mM HCl required to lower the pH to a target value of 3.45 for viral inactivation was equal to 0.305 times the start volume, then the composition of that process intermediate at pH 3.45 would be known from the dilution. Acetate, lactate, and NaOH would be present at 1/1.305 times their respective initial values (i.e., ˜15.3 mM acetate, ˜3.8 mM lactate, and ˜6.2 mM NaOH) and HCl present at 0.305/1.305 of its value in the titrant (-4.7 mM HC1). Similarly, for neutralization with 250 mM Tris base, if the ratio to raise the pH to a target of pH 7.0 was 0.0743 times the volume of pH 3.45 solution, ratios of 1/1.0743 and 0.0743/1.0743 would be applied to find the final concentrations in the neutralized solution (-14.3 mM acetate, ˜3.6 mM lactate, ˜5.8 mM NaOH, ˜4.4 mM HCl, and ˜17.3 mM Tris). All known values are plugged into the system of equations (Formulas 5 thru 15) to calculate the ionic strength:

$$I = \frac{1}{2} ( [ {H}^{+} ] \cdot \{ {1}^{2} \} + [ {Na}^{+} ] \cdot \{ {1}^{2} \} + [ {Trish H}^{+} ] \cdot \{ {1}^{2} \} + \qquad ( 5 )$$

$$[ {OH}^{-} ] \cdot {\{ - 1 \}}^{2} + [ {Acetate}^{-} ] \cdot {\{ - 1 \}}^{2} + [ {Lactate}^{-} ] \cdot {\{ - 1 \}}^{2} + [ {Cl}^{-} ] \cdot {\{ - 1 \}}^{2}$$

$$[ {H}^{+} ] + [ {Na}^{+} ] + [ {Tris H}^{+} ] = [ {OH}^{-} ] + {[ Acetate}^{-} ] + {[ Acetate}^{-} ] + [ {Cl}^{-} ] \qquad ( 6 )$$

$${K}_{a , Tris} = \frac{[ {H}^{+} ] [ Tris ]}{[ {Tris H}^{+} ]} \qquad ( 7 )$$

$${pK}_{a , Tris} = p {K}_{a , 0 , Tris} + 0.51 ( 2 \cdot \{ + 1 \} - 1 ) ( \frac{\sqrt{I}}{1 + \sqrt{I}} - 0.3 \sqrt{I} ) \qquad ( 8 )$$

$${K}_{a , Acetate} = \frac{[ {H}^{+} ] [ {Acetate}^{-} ]}{[ H Acetate ]} \qquad ( 9 )$$

$${PK}_{a , Acetate} = {pK}_{a , 0 , Acetate} + 0.51 ( 2 \cdot \{ - 1 \} - 1 ) ( \frac{\sqrt{I}}{1 + \sqrt{I}} - 0.3 \sqrt{I} ) \qquad ( 10 )$$

$${K}_{a , Lactate} = \frac{[ {H}^{+} ] [ {Lactate}^{-} ]}{[ H Lactate ]} \qquad ( 11 )$$

$${PK}_{a , Acetate} = {pK}_{a , 0 , Acetate} + 0.51 ( 2 \cdot \{ - 1 \} - 1 ) ( \frac{\sqrt{I}}{1 + \sqrt{I}} - 0.3 \sqrt{I} ) \qquad ( 12 )$$

$$Total Tris = [ T r i s ] + [ {Tris H}^{+} ] \qquad ( 13 )$$

$$Total Acetate = [ H Acetate ] + [ {Acetate}^{-} ] \qquad ( 14 )$$

$$Total Lactate = [ H Lactate ] + [ {Lactate}^{-} ] \qquad ( 15 )$$

where respective pK_{a,o} value for Tris, acetate, and lactate were taken to be 8.15, 4.76, and 3.86 at 22 ° C. The resulting estimate for the ionic strength of the depth filtration feed material is 22.1 mM.

As described herein, buffering capacity of a protein product is not directly modeled. Thus, when using a strong acid or base for titration, some deviations can arise between calculations and empirical titration results. For example, when titrating a Protein A eluate to low pH for viral inactivation, the buffer calculations typically underestimate the empirical amount of 20 mM HCl needed; the empirical amount needed may be on the order of 50% greater than the calculated estimate. One way to account for this difference is to model the affinity column eluate material at a higher pH, empirically adjusting the value until the estimated titration volume matches the experimental value. For example, in the above example, if the amount of 20 mM HCl was 50% higher than the 0.305 ratio than initially estimated, the Protein A eluate would be modeled as being about pH 4.45 instead of pH 4.2. Making this empirical change to the modeling, the estimated ionic strength in the example is directionally reduced, but only by a small amount: 21.9 mM down from the initial 22.1 mM estimate. Accordingly, it is concluded that either approach is sufficient for estimating ionic strength to deduce preferred embodiments of the present disclosure.

Alternative methods: Ion content measurement methods can be used to determine the buffer composition of the depth filtration feed material to calculate the ionic strength. This requires confirming that the measurements give self-consistent results with any known amounts such as the amounts of titrant added. Since the buffer composition of the affinity column eluate is assumed to be equivalent to that of the eluent but at a different pH, the difference in true composition could be determined by ion content measurements. For example, either an amount based on the eluent composition, or a measured value may be used to calculate ionic strength of the buffer components in the eluent.

### SEQUENCES

The following nucleic and/or amino acid sequences are referred to in the disclosure and are provided below for reference.

bamlanivimab variable heavy chain (VH)
SEQ ID NO: 1
QVQLVQSGAEVKKPGSSVKVSCKASGGTFSNYAISWVRQA
PGQGLEWMGRIIPILGIANYAQKFQGRVTITADKSTSTAY
MELSSLRSEDTAVYYCARGYYEARHYYYYYAMDVWGQGTA
VTVSS
SEQ ID NO: 2
bamlanivimab variable light chain (VL)
DIQMTQSPSSLSASVGDRVTITCRASQSISSYLSWYQQKP
GKAPKLLIYAASSLQSGVPSRFSGSGSGTDFTLTITSLQP
EDFATYYCQQSYSTPRTFGQGTKVEIK
bamlanivimab heavy chain (HC)
SEQ ID NO: 3
QVQLVQSGAEVKKPGSSVKVSCKASGGTFSNYAISWVRQA
PGQGLEWMGRIIPILGIANYAQKFQGRVTITADKSTSTAY
MELSSLRSEDTAVYYCARGYYEARHYYYYYAMDVWGQGTA
VTVSSASTKGPSVFPLAPSSKSTSGGTAALGCLVKDYFPE
PVTVSWNSGALTSGVHTFPAVLQSSGLYSLSSVVTVPSSS
LGTQTYICNVNHKPSNTKVDKRVEPKSCDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSREEMTKNQVSLTCLVKGFYPSDIAVEWESNGQPENNYK
TTPPVLDSDGSFFLYSKLTVDKSRWQQGNVFSCSVMHEAL
HNHYTQKSLSLSPGK
bamlanivimab light chain (LC)
SEQ ID NO: 4
DIQMTQSPSSLSASVGDRVTITCRASQSISSYLSWYQQKP
GKAPKLLIYAASSLQSGVPSRFSGSGSGTDFTLTITSLQP
EDFATYYCQQSYSTPRTFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC
etesevimab variable heavy chain (VH)
SEQ ID NO: 5
EVQLVESGGGLVQPGGSLRLSCAASGFTVSSNYMSWVRQA
PGKGLEWVSVIYSGGSTFYADSVKGRFTISRDNSMNTLFL
QMNSLRAEDTAVYYCARVLPMYGDYLDYWGQGTLVTVSS
etesevimab variable light chain (VL)
SEQ ID NO: 6
DIVMTQSPSSLSASVGDRVTITCRASQSISRYLNWYQQKP
GKAPKLLIYAASSLQSGVPSRFSGSGSGTDFTLTISSLQP
EDFATYYCQQSYSTPPEYTFGQGTKLEIKRTV
etesevimab heavy chain (HC)
SEQ ID NO: 7
EVQLVESGGGLVQPGGSLRLSCAASGFTVSSNYMSWVRQA
PGKGLEWVSVIYSGGSTFYADSVKGRFTISRDNSMNTLFL
QMNSLRAEDTAVYYCARVLPMYGDYLDYWGQGTLVTVSSA
STKGPSVFPLAPSSKSTSGGTAALGCLVKDYFPEPVTVSW
NSGALTSGVHTFPAVLQSSGLYSLSSVVTVPSSSLGTQTY
ICNVNHKPSNTKVDKRVEPKSCDKTHTCPPCPAPEAAGGP
SVFLFPPKPKDTLMISRTPEVTCVVVDVSHEDPEVKFNWY
VDGVEVHNAKTKPREEQYNSTYRVVSVLTVLHQDWLNGKE
YKCKVSNKALPAPIEKTISKAKGQPREPQVYTLPPSREEM
TKNQVSLTCLVKGFYPSDIAVEWESNGQPENNYKTTPPVL
DSDGSFFLYSKLTVDKSRWQQGNVFSCSVMHEALHNHYTQ
KSLSLSPGK
etesevimab light chain (LC)
SEQ ID NO: 8
DIVMTQSPSSLSASVGDRVTITCRASQSISRYLNWYQQKP
GKAPKLLIYAASSLQSGVPSRFSGSGSGTDFTLTISSLQP
EDFATYYCQQSYSTPPEYTFGQGTKLEIKRTVAAPSVFIF
PPSDEQLKSGTASVVCLLNNFYPREAKVQWKVDNALQSGN
SQESVTEQDSKDSTYSLSSTLTLSKADYEKHKVYACEVTH
QGLSSPVTKSFNRGEC
bebtelovimab variable heavy chain (VH)
SEQ ID NO: 9
QITLKESGPTLVKPTQTLTLTCTFSGFSLSISGVGVGWLR
QPPGKALEWLALIYWDDDKRYSPSLKSRLTISKDTSKNQV
VLKMTNIDPVDTATYYCAHHSISTIFDHWGQGTLVTVSS
SEQ ID NO: 10
bebtelovimab variable light chain (VL)
QSALTQPASVSGSPGQSITISCTATSSDVGDYNYVSWYQQ
HPGKAPKLMIFEVSDRPSGISNRFSGSKSGNTASLTISGL
QAEDEADYYCSSYTTSSAVFGGGTKLTVL
bebtelovimab heavy chain (HC)
SEQ ID NO: 11
QITLKESGPTLVKPTQTLTLTCTFSGFSLSISGVGVGWLR
QPPGKALEWLALIYWDDDKRYSPSLKSRLTISKDTSKNQV
VLKMTNIDPVDTATYYCAHHSISTIFDHWGQGTLVTVSSA
STKGPSVFPLAPSSKSTSGGTAALGCLVKDYFPEPVTVSW
NSGALTSGVHTFPAVLQSSGLYSLSSVVTVPSSSLGTQTY
ICNVNHKPSNTKVDKRVEPKSCDKTHTCPPCPAPELLGGP
SVFLFPPKPKDTLMISRTPEVTCVVVDVSHEDPEVKFNWY
VDGVEVHNAKTKPREEQYNSTYRVVSVLTVLHQDWLNGKE
YKCKVSNKALPAPIEKTISKAKGQPREPQVYTLPPSREEM
TKNQVSLTCLVKGFYPSDIAVEWESNGQPENNYKTTPPVL
DSDGSFFLYSKLTVDKSRWQQGNVFSCSVMHEALHNHYTQ
KSLSLSPGK
bebtelovimab light chain (LC)
SEQ ID NO: 12
QSALTQPASVSGSPGQSITISCTATSSDVGDYNYVSWYQQ
HPGKAPKLMIFEVSDRPSGISNRFSGSKSGNTASLTISGL
QAEDEADYYCSSYTTSSAVFGGGTKLTVLGQPKAAPSVTL
FPPSSEELQANKATLVCLISDFYPGAVTVAWKADSSPVKA
GVETTTPSKQSNNKYAASSYLSLTPEQWKSHRSYSCQVTH
EGSTVEKTVAPTECS

1. A method of reducing host cell protein content in a protein preparation comprising a protein of interest recombinantly produced in a host cell, the method comprising the steps of:
a. subjecting the protein preparation to an affinity chromatography column;
b. eluting the protein of interest from the chromatography column with a combination of acids comprising of a weak acid and a strong acid to obtain an eluate comprising the protein of interest;
c. raising pH of the eluate to above about pH 6.0; and
d. subjecting the eluate to a depth filter and obtaining a filtered protein preparation.

2. The method of claim 1, wherein the chromatography column comprises a Protein A, Protein G or Protein L affinity chromatography column.

3. The method of claim 1, wherein the weak acid has no more than one pKa value less than 7.0, and the strong acid has no more than one pKa value less than 7.0.

4. The method of claim 1, wherein the weak acid is acetic acid and the strong acid is phosphoric acid or lactic acid.

5. The method of claim 4, wherein the concentration of the acetic acid is about 20 mM, and wherein the strong acid is phosphoric acid and wherein the concentration of the phosphoric acid is about 5 mM to about 10 mM.

6. The method of claim 4, wherein the concentration of the acetic acid is about 20 mM, and wherein the strong acid is lactic acid and wherein the concentration of the lactic acid is about 5 mM.

7. The method of claim 1, further comprising a step of performing viral inactivation.

8. The method of claim 1, further comprising a step of performing viral inactivation, comprising adjusting the pH of the eluate from said step of eluting the protein from the chromatography column, to below about pH 4.0, and wherein the eluate is maintained at below about pH 4.0 for about 0 minutes to about 180 minutes.

9. The method of claim 8, wherein said step of adjusting the pH of the eluate comprises adjusting the pH of the eluate to about pH 3.3 to about pH 3.7

10. The method of claim 9, wherein the pH of the eluate is adjusted to about pH 3.5.

11. The method of claim 8, wherein adjusting the pH of the eluate comprises adding any one of HCl, phosphoric acid, or a combination of acetic acid and phosphoric acid.

12. The method of claim 1, wherein said step of raising the pH of the eluate comprises raising the pH to about pH 6.5 to about pH 7.5.

13. The method of claim 12, wherein the pH of the eluate is raised to about pH 7.0.

14. The method of claim 12, wherein the step of raising the pH of the eluate comprises adding Tris.

15. The method of claim 1, wherein the eluate at said step of raising the pH to above about 6.0 has an ionic strength of about 10 mM to about 45 mM.

16. The method of claim 1, further comprising a step of subjecting the depth filtered protein preparation to ion exchange chromatography.

17. The method of claim 1, wherein the host cell protein content in the filtered protein preparation is reduced to less than 100 ppm.

18. The method of claim 1, wherein the host cell protein content in the filtered protein preparation comprises PLBL2, and wherein the PLBL2 is reduced to less than 100 ppm.

19. The method of claim 1, wherein the protein preparation comprises a harvested cell culture fluid, a capture pool, or a recovered protein pool.

20. The method of claim 1, wherein the protein is a therapeutic or diagnostic protein.

21. The method of claim 1, wherein the protein is an antibody, Fc Fusion protein, peptide, an immunoadhesin, an enzyme, a growth factor, a receptor, a hormone, a regulatory factor, a cytokine, an antigen, a peptide, or a binding agent.

22. The method of claim 21, wherein the antibody is a monoclonal antibody, a chimeric antibody, a humanized antibody, a human antibody, a bispecific antibody, or an antibody fragment.

23. The method of claim 22, wherein the antibody is an IgG1 antibody.

24. The method of claim 1, wherein the protein is an anti-SARS-COV-2 antibody.

25. The method of claim 24, wherein the anti-SARS-COV-2 antibody is bamlanivimab.

26. The method of claim 24, wherein the anti-SARS-COV-2 antibody comprises a VH of SEQ ID NO: 1 and a VL of SEQ ID NO: 2.

27. The method of claim 24, wherein the anti-SARS-COV-2 antibody comprises a HC of SEQ ID NO: 3 and a LC of SEQ ID NO: 4.

28. The method of claim 24, wherein the anti-SARS-COV-2 antibody is etesevimab.

29. The method of claim 24, wherein the anti-SARS-COV-2 antibody comprises a VH of SEQ ID NO: 5 and a VL of SEQ ID NO: 6.

30. The method of claim 24, wherein the anti-SARS-COV-2 antibody comprises a HC of SEQ ID NO: 7 and a LC of SEQ ID NO: 8.

31. The method of claim 24, wherein the anti-SARS-COV-2 antibody is bebtelovimab.

32. The method of claim 24, wherein the anti-SARS-COV-2 antibody comprises a VH of SEQ ID NO: 9 and a VL of SEQ ID NO: 10.

33. The method of claim 24, wherein the anti-SARS-COV-2 antibody comprises a HC of SEQ ID NO: 11 and a LC of SEQ ID NO: 12.

34. A method of reducing host cell protein content in an anti-SARS-COV-2 antibody preparation recombinantly produced in a host cell comprising:
a. subjecting the anti-SARS-COV-2 antibody preparation recombinantly produced in a host cell to a Protein A affinity chromatography column;
b. eluting the anti-SARS-COV-2 antibody with a combination of acids comprising of acetic acid and phosphoric acid or a combination of acetic acid and lactic acid to obtain an eluate comprising the anti-SARS-COV-2 antibody;
c. adjusting the pH of the eluate comprising the anti-SARS-COV-2 antibody by addition of about 20 mM HCl, wherein the pH is lowered to about pH 3.3 to about pH 3.7, and wherein the eluate is maintained at about pH 3.3 to about pH 3.7 for about 0 minutes to about 180 minutes;
d. raising the pH of the eluate comprising the anti-SARS-COV-2 antibody by addition of about 250 mM Tris Buffer, wherein the pH is raised to about pH 6.5 to about pH 7.5; and
e. subjecting the eluate comprising the anti-SARS-COV-2 antibody to a depth filter, and obtaining a filtered anti-SARS-COV-2 antibody preparation, wherein host cell protein content in the filtered anti-SARS-COV-2 antibody preparation is reduced to about 0 ppm to about 20 ppm, and wherein the anti-SARS-COV-2 antibody is an IgG1 antibody.

35. The method of claim 34, wherein the combination of acids of step b comprises 20 mM acetic acid and 5 mM phosphoric acid, or 20 mM acetic acid and 5 mM phosphoric acid, or 20 mM acetic acid and 5 mM lactic acid.

36. The method of claim 34, wherein step c of adjusting the pH of the eluate comprises adjusting the pH of the eluate to about 3.5.

37. The method of claim 34, wherein said step of adjusting the pH of the eluate comprising the anti-SARS-COV-2 antibody by addition of about 20 mM HCl achieves viral inactivation.

38. The method of claim 34, wherein said step of raising the pH of the eluate comprises raising said pH to about pH 7.25.

39. The method of claim 34, wherein the eluate after said step of raising the pH has an ionic strength of about 10 mM to about 45 mM.

40. The method of claim 34, further comprising a step of subjecting the depth filtered anti-SARS-COV-2 antibody preparation to ion exchange chromatography.

41. The method of claim 34, wherein the anti-SARS-COV-2 antibody is bamlanivimab.

42. The method of claim 34, wherein the anti-SARS-COV-2 antibody comprises a VH of amino acid SEQ ID NO: 1 and a VL of amino acid SEQ ID NO: 2.

43. The method of claim 34, wherein the anti-SARS-COV-2 antibody comprises a HC of amino acid SEQ ID NO: 3 and a LC of amino acid SEQ ID NO: 4.

44. The method of claim 34, wherein the anti-SARS-COV-2 antibody is etesevimab.

45. The method of claim 34, wherein the anti-SARS-COV-2 antibody comprises a VH of SEQ ID NO: 5 and a VL of SEQ ID NO: 6.

46. The method of claim 34, wherein the anti-SARS-COV-2 antibody comprises a HC of SEQ ID NO: 7 and a LC of SEQ ID NO: 8.

47. The method of claim 34, wherein the anti-SARS-COV-2 antibody is bebtelovimab.

48. The method of claim 34, wherein the anti-SARS-COV-2 antibody comprises a VH of amino acid SEQ ID NO: 9 and a VL of amino acid SEQ ID NO: 10.

49. The method of claim 34, wherein the anti-SARS-COV-2 antibody comprises a HC of amino acid SEQ ID NO: 11 and a LC of amino acid SEQ ID NO: 12.

50. The method of claim 34, wherein the depth filter comprises C0SP, X0SP, X0HC, Emphaze AEX Hybrid Purifier, or Zeta Plus (ZB Media).

51. The method of claim 1, wherein the host cell is a mammalian cell.

52. The method of claim 51, wherein the mammalian cell is a CHO cell.

53. A composition produced by the method of claim 1.

54. The composition of claim 53, wherein the host cell protein content in the composition is less than about 100 ppm.

55. The method of claim 34, wherein the host cell is a mammalian cell.

56. The method of claim 55, wherein the mammalian cell is a CHO cell.

57. A composition produced by the method of claim 34.

58. The composition of claim 57, wherein the host cell protein content in the composition is less than about 100 ppm.


SEQUENCE LISTING (12 sequences)

SEQ ID NO: 1 - protein, 125 aa, Artificial Sequence
  Synthetic Construct
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSS

SEQ ID NO: 2 - protein, 107 aa, Artificial Sequence
  Synthetic Construct
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIK

SEQ ID NO: 3 - protein, 455 aa, Artificial Sequence
  Synthetic Construct
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Synthetic Construct
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 119 aa, Artificial Sequence
  Synthetic Construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSS

SEQ ID NO: 6 - protein, 112 aa, Artificial Sequence
  Synthetic Construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTV

SEQ ID NO: 7 - protein, 449 aa, Artificial Sequence
  Synthetic Construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial Sequence
  Synthetic Construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 119 aa, Artificial Sequence
  Synthetic Construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSS

SEQ ID NO: 10 - protein, 109 aa, Artificial Sequence
  Synthetic Construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVL

SEQ ID NO: 11 - protein, 449 aa, Artificial Sequence
  Synthetic Construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  Synthetic Construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS